(19) United States
(12) Patent Application Publication
(10) Pub. No.: US 2025/0147491 A1
(43) Pub. Date: May 8, 2025

(54) IOT-ENABLED SYSTEMS AND METHODS FOR MONITORING METHANE EMISSIONS OF ONE OR MORE INDUSTRIAL FACILITIES

(71) Applicant: Schlumberger Technology Corporation, Sugar Land, TX (US)

(72) Inventors: Raphael M. Gadot, Sugar Land, TX (US); Adam P. Huynh, Richmond, TX (US)

(21) Appl. No.: 18/833,538

(22) PCT Filed: Apr. 3, 2023
(86) PCT No.: PCT/US2023/017298

§ 371 Date: Jul. 26, 2024

Related U.S. Application Data
(60) Provisional application No. 63/362,548, filed on Apr. 6, 2022.

(51) Int. Cl. G05B 19/418; G01N 33/00

(52) U.S. Cl. CPC G05B 19/4186; G01N 33/0075; G05B 19/41885

(57) ABSTRACT

The disclosure relates to a system for monitoring methane emissions at industrial facilities. The system includes a network of methane emission detectors at different locations within at least one industrial facility; a gateway device operably coupled to the network of methane emission detectors; and a cloud computing environment operably coupled to the gateway device; wherein the network of methane emission detectors is configured to perform time-series measurements at different locations within the at least one industrial facility and wirelessly communicate time-series sensor data representing such measurements to the gateway device; wherein the gateway device is configured to process the time-series sensor data to derive time-series operational data and communicate the time-series operational data to the cloud computing environment; and wherein the cloud computing environment is configured to receive and process the time-series operational data to detect and characterize methane emission at the at least one industrial facility.

### CROSS-REFERENCE TO RELATED APPLICATION(S)

The subject disclosure claims priority from U.S. Provisional Appl. No. 63/362,548, filed on Apr. 6, 2022, entitled “IOT-ENABLED SYSTEMS AND METHODS FOR MONITORING METHANE EMISSIONS OF ONE OR MORE INDUSTRIAL FACILITIES”, herein incorporated by reference in its entirety.

### FIELD

The subject disclosure relates to systems and methods for monitoring methane emissions at industrial facilities, such as oil and gas facilities including well sites, compressor stations, and processing facilities

### BACKGROUND

Methane emissions in the oil and gas industry are receiving intense scrutiny as it is believed that such methane emissions contribute to global warming and/or climate change. A large portion of the methane emissions in the oil and gas industry arise from a small number of major emission events henceforth referred to as super-emitters. Super-emitters occur from a variety of sites, and recent data suggest many emission events are intermittent.

Continuous monitoring using methane emission detectors installed permanently at a site offers an effective way to identify, quantify, and mitigate intermittent methane emissions. However, installing methane emission detectors across many diverse sites can be economically challenging.

### SUMMARY

In embodiments, automated systems and methods are provided for methane emissions monitoring of one or more industrial facilities. The automated systems and methods can be economically deployed worldwide on a large scale. The automated systems and methods employ a network of methane emission detectors spaced from one another at different locations at the one or more industrial facilities. The methane emission detectors of the network are configured to perform time-series measurements of methane concentration at different locations within the one or more industrial facilities and wirelessly communicate time-series sensor data representing such measurements (i.e., high-frequency location-specific raw methane concentration data) to an edge gateway device. The edge gateway can be located at or near the one or more industrial facilities or within communication range from the one or more industrial facilities.

One or more methane emission detectors of the network can also include one or more environmental sensors that are configured to perform time-series measurements of environmental conditions (such as wind speed, wind direction, temperature, humidity, atmospheric pressure, and solar radiation) at one or more locations within the industrial facility(ies). The one or more methane emission detectors with environmental sensor(s) are further configured to wirelessly communicate time-series sensor data representing such measurements (i.e., high-frequency location-specific raw environmental data) to the edge gateway device.

Each methane emission detector of the network can include a Global Navigation Satellite System (GNSS) module that precisely tracks the position of the methane emission detector and time. The sensor data communicated from a given methane emission detector of the network can be stamped with location and time information as recorded by the Global Navigation Satellite System (GNSS) module in synchronization with the time-series measurements performed by the given methane emission detector.

The edge gateway device can be configured to collect, aggregate, and process the time-series sensor data (i.e., the high-frequency location-specific raw methane concentration data and the high-frequency location-specific raw environmental data) communicated from the network of methane emission detectors, for example, by filtering and/or averaging the sensor data to derive corresponding time-series operational data and forwards such time-series operational data to a remote cloud computing environment. The time-series operational data can represent i) methane concentration at specific locations within the one or more industrial facilities as a function of time as derived from the location-specific raw methane concentration data, and ii) environmental conditions at specific location(s) within the one or more industrial facilities as a function of time as derived from the location-specific raw environmental data.

The remote cloud computing environment can be configured to receive the time-series operational data communicated from the edge gateway device and process the received time-series operational data using a suitable computational model, such as Gaussian plume dispersion model, to characterize methane emission at the one or more industrial facilities. For example, the cloud computing environment can process the received time-series operational data using a suitable computational model (e.g., Gaussian plume dispersion model) to detect the presence of methane emission at the one or more industrial facilities, the location of the methane emission at the one or more industrial facilities (when present), and the associated rate of methane emission at the one or more industrial facilities (when present).

The cloud computing environment can be configured to generate data related to the methane emission (such as the location of the methane emission at the one or more industrial facilities, and the associated rate of methane emission at the one or more industrial facilities) and process such data to automatically generate an alert characterizing the methane emission at the one or more industrial facilities. The alert can be communicated to a designated party or user to initiate or schedule mitigation of the methane emission at the one or more industrial facilities.

In embodiments, each methane emission detector of the network can include a methane sensor for measuring methane concentration in the immediate vicinity of the sensor together with the electronics that interface with the methane sensor and provide for wireless data communication between the methane emission detector and the edge gateway device. In embodiments, the wireless data communication can implement a predefined wireless communication protocol, such as the LoRaWAN protocol. The LoRaWAN protocol employs spread spectrum modulation techniques derived from chirp spread spectrum (CSS) technology and provides a range up to 10 km with a data rate of up to 50 kbps.

In embodiments, each methane emission detector of the network can be configured to wirelessly communicates time-series sensor data directly to the gateway device. Alternatively, the network of methane emission detectors can be configured as a wireless mesh sensor network wherein time-series sensor data is wirelessly communicated to a nearby methane emission detector for forwarding to the gateway device.

In embodiments, each methane emission detector of the network can include an accelerometer for measuring the acceleration of the methane emission detector. Detector-specific raw acceleration data can be communicated from the methane emission detectors of the network to the edge gateway device and processed by the edge gateway to detect fall events for the respective methane emission detectors. Additionally or alternatively, detector-specific acceleration data can be derived from such measurements by the edge gateway device and communicated from the edge gateway device to the cloud computing environment. The cloud computing environment can be configured to receive and process the detector-specific acceleration data to detect fall events for the respective methane emission detectors.

In embodiments, one or more methane emission detectors of the network can include environmental sensors selected from the group that includes: an anemometer for measuring wind speed and wind direction, a pyranometer for measuring solar radiation, one or more sensors that measure temperature and humidity, and a barometer for measuring atmospheric pressure.

In embodiments, the network of methane emission detectors can be deployed over locations within one or more oil and gas facilities, such as one or more well sites, one or more compressor stations, or one or more processing facilities.

In embodiments, the edge gateway device can be configured to collect and process time-series sensor data measured by methane emission detectors at multiple industrial facilities (e.g., multiple well sites), and the cloud computing environment can be configured to process time-series operational data derived from the time-series sensor data measured by the methane emission detectors at the multiple industrial facilities to characterize methane emission at the respective industrial facilities.

In other embodiments, the methane emission detectors can be configured to process the real-time measurement data, for example, including filtering and/or averaging, and forward the resultant time-series data to the gateway device. The gateway device can collect, aggregate, and process such data for communication to the cloud computing environment.

In still other embodiments, the gateway device can be configured to process time-series operational data derived from the measurements performed by the network of methane emission detectors wirelessly coupled thereto using a suitable computational model, such as Gaussian plume dispersion model, to characterize methane emission at one or more industrial facilities. For example, the gateway device can process the time-series operational data using a suitable computational model (e.g., simulation and inversion using a Gaussian plume dispersion model) to detect the presence of methane emission at one or more industrial facilities, the location of the methane emission at one or more industrial facilities (when present), and the associated rate of methane emission at the one or more industrial facilities (when present). The gateway device can be further configured to generate data related to the methane emission (such as the location of the methane emission at the one or more industrial facilities, and the associated rate of methane emission at the one or more industrial facilities) and process such data to automatically generate alert(s) characterizing the methane emission at the one or more industrial facilities. The alert(s) can be communicated to designated party(ies) or user(s) to initiate or schedule mitigation of the methane emission at the one or more industrial facilities.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF DRAWINGS

The subject disclosure is further described in the detailed description which follows, in reference to the noted plurality of drawings by way of non-limiting examples of the subject disclosure, in which like reference numerals represent similar parts throughout the several views of the drawings, and wherein:

FIG. 1 is a schematic block diagram that depicts an automated system for methane emissions monitoring in accordance with an embodiment of the subject disclosure;

FIG. 2 is a schematic block diagram that depicts another automated system for methane emissions monitoring in accordance with an embodiment of the subject disclosure;

FIGS. 3A and 3B, collectively, is a flow chart of operations that can be carried out by the cloud computing environment of FIGS. 1 and 2;

FIG. 4 is a schematic diagram that depicts the automated system for methane emissions monitoring of FIG. 1 deployed at a well site (e.g., well pad);

FIG. 5 is a schematic diagram that depicts the automated system for methane emissions monitoring of FIG. 1 deployed at another well site (e.g., well pad); and

FIG. 6 is a schematic diagram of an example computing system.

### DETAILED DESCRIPTION

The particulars shown herein are by way of example and for purposes of illustrative discussion of the embodiments of the subject disclosure only and are presented in the cause of providing what is believed to be the most useful and readily understood description of the principles and conceptual aspects of the subject disclosure. In this regard, no attempt is made to show structural details in more detail than is necessary for the fundamental understanding of the subject disclosure, the description taken with the drawings making apparent to those skilled in the art how the several forms of the subject disclosure may be embodied in practice. Furthermore, like reference numbers and designations in the various drawings indicate like elements.

The present disclosure is directed to automated systems and methods that provide for methane emissions monitoring of one or more industrial facilities. The automated systems and methods can be economically deployed worldwide on a large scale.

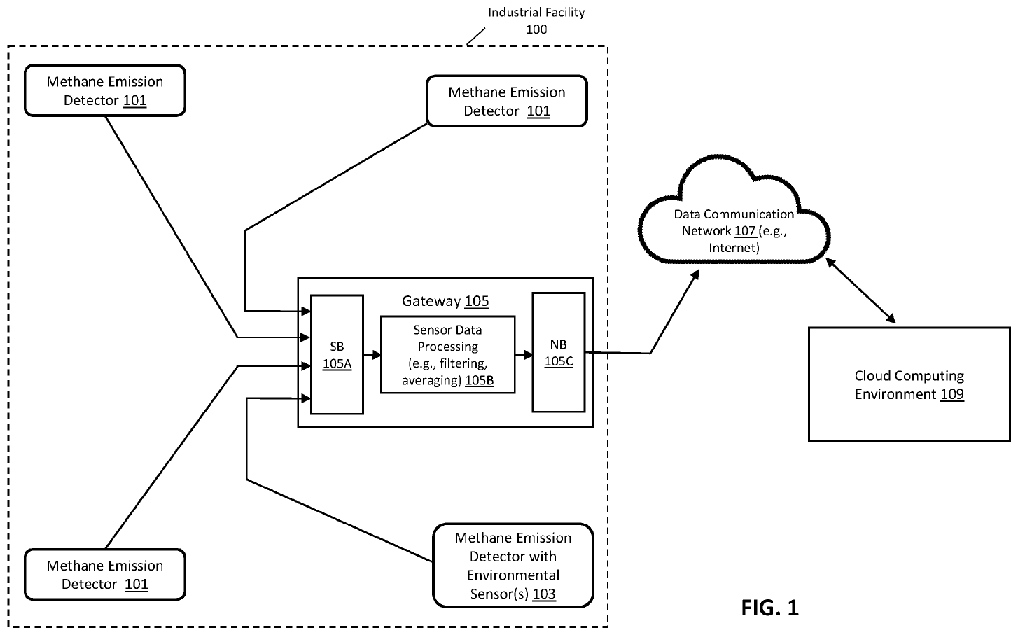

In an example embodiment shown in FIG. 1, an automated system is provided that includes an edge gateway device 105 that is located at or near (e.g., at a distance of 10 km or less) an industrial facility 100. The edge gateway device 105 is a ruggedized computing device that can be configured to deliver performance edge computing and secure data ingestion. The edge gateway device 105 can be configured to enable real-time monitoring and control of the operational equipment at the industrial facility 100.

The edge gateway device 105 can be configured to receive, collect, and aggregate data from a variety of operational equipment at the industrial facility 100 (such as sensors, controllers, actuators, programmable logic controllers, remote terminal units, and supervisory control and data acquisition (SCADA) systems), prepare such data for transmission to the remote cloud computing environment 109, and transmit the data from the edge gateway device 105 to the remote cloud computing environment 109 over a data communication network 107 as shown in FIG. 1. The data communication network 107 can include a cellular data network, satellite link, other Wide Area Network, the Internet, and/or another mode of available data communication. The cloud computing environment 109 can be implemented by one or more processor-based systems as is well known.

In embodiments, the edge gateway device 105 can employ a compact and rugged NEMA/IP rated housing for outdoor use, making it suitable for the environments at well sites and facilities. The overall packaging can also be environmentally qualified.

In embodiments, the gateway device 105 can be configured with a bi-directional communication interface (referred to as a Southbound Interface labeled SB, 105A) for data communication to the operational equipment at the facility 100 using either a wired communication protocol (such as a serial, Ethernet, Modbus or Open Platform Communication (OPC) protocol) or a wireless communication protocol (such as IEEE 802.11 Wi-Fi protocol, Highway Addressable Remote Transducer Protocol (HART), LoraWAN, WiFi or Message Queuing Telemetry Transport (MQTT)). The Southbound Interface 105A can provide for direct data communication to the operational equipment at the facility 100. Alternatively, the Southbound Interface 105A can provide for indirect data communication to the operational equipment at the facility 100 via a local area network or other local communication devices.

In embodiments, the edge gateway device 105 can be configured with a bi-directional communication interface (referred to as a Northbound Interface labeled NB, 105C) to the data communication network 107 using a wireless communication protocol. In embodiments, the wireless communication protocol can employ cellular data communication, such as 4G LTE data transmission capability (or possibly 3G data transmission for fallback capability). For facilities without a cellular signal, the Northbound Interface 105C to the data communication network 107 can be provided by a bidirectional satellite link (such as a BGAN modem). Alternatively, the Northbound Interface 105C can implement other wireless communication protocols or wired communication protocols implemented by the data communication network 107.

In embodiments, the edge gateway device 105 can employ an embedded processing environment (e.g., data processor and memory system) that hosts and executes an operating system and application(s) or module(s) as described herein.

In embodiments, the edge gateway device 105 can employ both hardware-based and software-based security measures. The hardware-based security measures can involve a hardware root-of-trust established using an industry-standard Trusted Platform Module (TPM) v2.0 cryptographic chip. The software-based security measures can include operating system hardening and encryption of both buffered and transmitted data.

In embodiments, the edge gateway device 105 can support a containerized microservice-based architecture. This architecture enables extensibility into several distinct and different solutions for different environments and applications at the edge, while still using the same infrastructure components. In embodiments, the edge gateway device 105 can employ one or more containers to implement one or more applications or modules executing on the gateway device 105 that perform functionality for methane emissions monitoring as described herein. A container is a standard unit of software that packages up code and all its dependencies (such as runtime environment, system tools, system libraries and settings) so that the application or module runs quickly and reliably in the computing environment of the edge gateway device 105. The container isolates the software from its environment and ensures that it works uniformly and reliably in the computing environment of the edge gateway device 105.

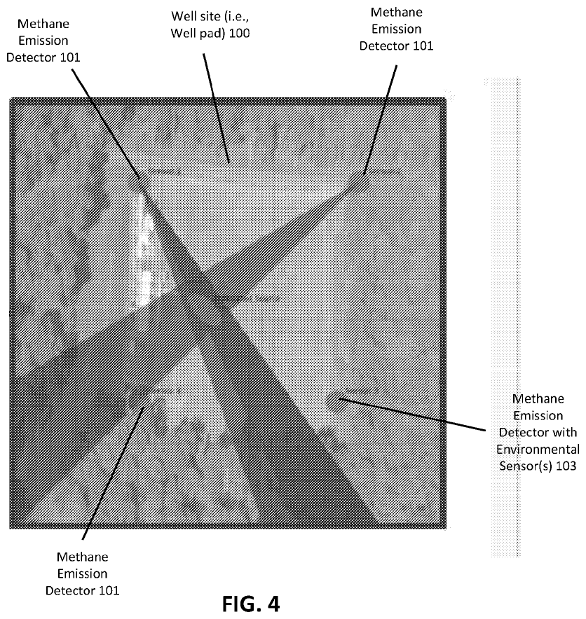

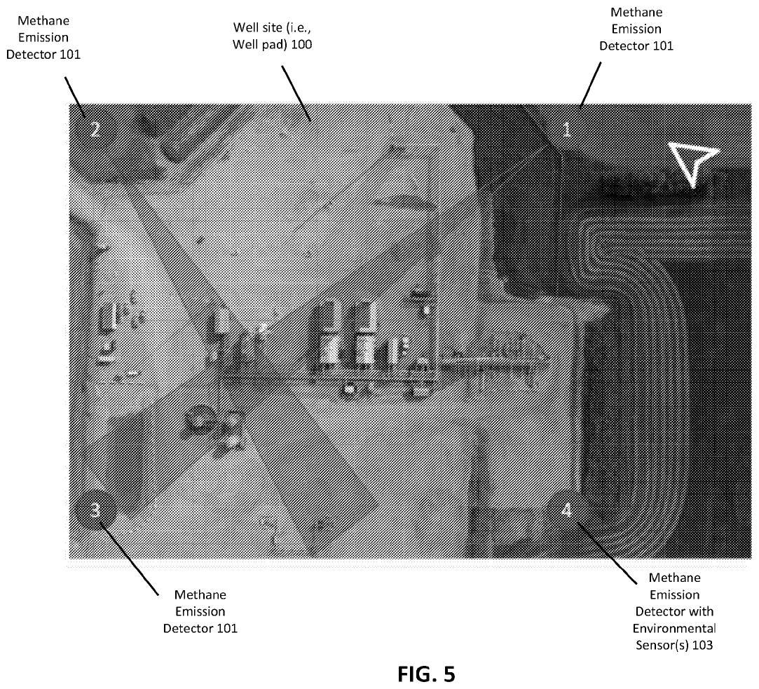

In embodiments, the Southbound Interface 105A of the gateway device 105 utilizes a wireless communication protocol to interface to a network of methane emission detectors (for example, three methane emission detectors 101 and one methane emission detector with environmental sensor(s) 103) that are spaced from one another at different locations at the industrial facility 100, for example in opposed corners of the facility as shown in FIGS. 4 and 5. The network of methane emission detectors 101, 103 is configured to perform time-series measurements of methane concentration at different locations within the industrial facility 100 and wirelessly communicate time-series sensor data representing such measurements (i.e., high-frequency location-specific raw methane concentration data) to the edge gateway device 105.

In embodiments, one or more methane emission detectors of the network, such as the methane emission detector 103 in FIG. 1, can include one or more environmental sensors that are configured to perform time-series measurements of environmental conditions (such as wind speed, wind direction, temperature, humidity, atmospheric pressure, and solar radiation) at one or more locations within the industrial facility 100. The one or more methane emission detectors with environmental sensor(s) can be further configured to wirelessly communicate time-series sensor data representing such measurements (i.e., high-frequency location-specific raw environmental data) to the edge gateway device 105. In embodiments, the environmental sensors can be selected from the group that includes: an anemometer for measuring wind speed and wind direction, a pyranometer for measuring solar radiation, one or more sensors that measure temperature and humidity, and a barometer for measuring atmospheric pressure.

In embodiments, each methane emission detector (101, 103) of the network can include a Global Navigation Satellite System (GNSS) module that precisely tracks the position of the methane emission detector and time. The time-series sensor data communicated from a given methane emission detector (101, 103) can be stamped with location and time information as recorded by the Global Navigation Satellite System (GNSS) module in synchronization with the time-series measurements performed by the given methane emission detector (101, 103).

In embodiments, each methane emission detector (101, 103) of the network can include a methane sensor for measuring methane concentration in the immediate vicinity of the methane sensor together with the electronics that interface to the methane sensor and provide for direct wireless data communication between the methane emission detector (101, 103) and the edge gateway device 105. In embodiments, the wireless data communication can implement a predefined wireless communication protocol, such as the LoRaWAN protocol. The LoRaWAN protocol employs spread spectrum modulation techniques derived from chirp spread spectrum (CSS) technology and provides a range up to 10 km with a data rate of up to 50 kbps. In embodiments, the methane emission detectors (101, 103) and the edge gateway device 105 can be arranged in a star network configuration where each one of the methane emission detectors (101, 103) directly communicates with the edge gateway device 105 over a wireless communication link that implements a predefined wireless communication protocol, such as the LoRaWAN protocol.

The edge gateway device 105 includes sensor data processing functionality 105B that is configured to collect, aggregate, and process the time-series sensor data (i.e., the high-frequency location-specific raw methane concentration data and the high-frequency location-specific raw environmental data) communicated from the network of methane emission detectors 101, 103, for example, by filtering and/or averaging the data, to generate corresponding time-series operational data. The time-series operational data represents i) methane concentration at specific locations within the industrial facility 100 as a function of time as derived from the location-specific raw methane concentration data, and ii) environmental conditions at specific location(s) within the industrial facility 100 as a function of time as derived from the location-specific raw environmental data. The sensor data processing functionality 105B is further configured to cooperate with the Northbound Interface 105C to forward the time-series operational data to the remote cloud computing environment 109 via the data communication network 107. The cloud computing environment 109 receives the time-series operational data communicated from the edge gateway device 105 and processes the received time-series operational data to detect the presence of methane emission at the industrial facility 100, the location of the methane emission at the industrial facility 100 (when present), and the associated rate of methane emission at the industrial facility 100 (when present). The cloud computing environment 109 can generate data related to the methane emission (such as the location of the methane emission at the industrial facility 100, and the associated rate of methane emission at the industrial facility 100) and process such data to automatically generate an alert characterizing the methane emission. The alert can be communicated to a designated party or user to initiate or schedule mitigation of the methane emission at the industrial facility 100.

In embodiments, the cloud computing environment 109 can employ a suitable computational model, such as Gaussian plume dispersion model, to detect the presence of methane emission at the industrial facility 100, the location of the methane emission at the industrial facility 100, and the associated rate of methane emission at the industrial facility 100. In this embodiment, the computational model can be used as part of simulation and inversion operations that detects the presence of methane emission at the industrial facility 100, the location of the methane emission at the industrial facility 100, and the associated rate of methane emission at the industrial facility 100.

In embodiments, each methane emission detector (101, 103) of the network can include an accelerometer for measuring the acceleration of the methane emission detector. Detector-specific raw acceleration data can be communicated from the methane emission detectors (101, 103) of the network to the edge gateway device 105 and processed by the edge gateway device 105 to detect fall events for the respective methane emission detectors 101, 103. Additionally or alternatively, the edge gateway device 105 can derive detector-specific acceleration data from such measurements, and communicate the detector-specific acceleration data from the edge gateway device 105 to the cloud computing environment 109. The cloud computing environment 109 can be configured to receive and process the detector-specific acceleration data to detect fall events for the respective methane emission detectors (101, 103) of the network. The alert can be communicated to a designated party or user to initiate or schedule repair of the fallen emission detector at the industrial facility 100.

In embodiments, the edge gateway device 105 can be configured to collect and process time-series sensor data measured by methane emission detector networks at multiple industrial facilities (e.g., multiple well sites), and the cloud computing environment 109 can be configured to process time-series operational data derived from the time-series sensor data measured by the methane emission detector networks at the multiple industrial facilities to characterize methane emission at the respective industrial facilities.

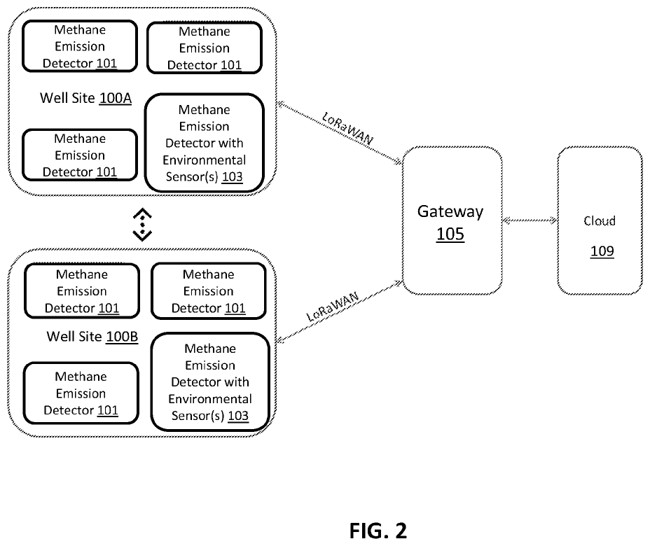

For example, in another example embodiment shown in FIG. 2, the edge gateway device 105 (e.g., the Southbound Interface 105A) utilizes a wireless communication protocol to interface to multiple networks of methane emission detectors for monitoring emissions for two well sites 100A and 100B as shown in FIG. 2. In this embodiment, the methane emission detectors of each network are spaced from one another at different locations within the respective well sites 100A, 100B. For example, the network of methane emission detectors (101, 103) for the well site 100A can be deployed in opposed corners of the well site 100A as shown in FIG. 4, while the network of methane emission detectors (101, 103) for the well site 100B can be deployed in opposed corners of another well site 100B as shown in FIG. 5. The multiple networks of methane emission detectors (101, 103) are configured to perform time-series measurements of methane concentration at different locations within the corresponding well sites 100A, 100B and wirelessly communicate time-series sensor data representing such measurements (i.e., high-frequency location-specific raw methane concentration data) to the edge gateway device 105.

In embodiments, one or more methane emission detectors of each network, such as the methane emission detector 103 for well site 100A and the methane emission detector 103 for well site 100B in FIG. 2, can include one or more environmental sensors that are configured to perform time-series measurements of environmental conditions (such as wind speed, wind direction, temperature, humidity, atmospheric pressure, and solar radiation) at one or more locations within the respective well sites 100A, 100B. The one or more methane emission detectors with environmental sensor(s), such as the methane emission detector 103 for well site 100A and the methane emission detector 103 for well site 100B, can be further configured to wirelessly communicate time-series sensor data representing such measurements of environmental conditions (i.e., high-frequency location-specific raw environmental data) to the edge gateway device 105. In embodiments, the environmental sensors can be selected from the group that includes: an anemometer for measuring wind speed and wind direction, a pyranometer for measuring solar radiation, one or more sensors that measure temperature and humidity, and a barometer for measuring atmospheric pressure.

In embodiments, each methane emission detector (101, 103) of the multiple networks can include a Global Navigation Satellite System (GNSS) module that precisely tracks the position of the methane emission detector and time. The time-series sensor data representing the time-series measurements communicated from a given methane emission detector (101, 103) can be stamped with location and time information as recorded by the Global Navigation Satellite System (GNSS) module in synchronization with the time-series measurements performed by the given methane emission detector (101, 103).

In embodiments, each methane emission detector (101, 103) of the multiple networks can include a methane sensor for measuring methane concentration in the immediate vicinity of the methane sensor together with the electronics that interface to the methane sensor and provide for wireless data communication between the methane emission detector (101, 103) and the edge gateway device 105. In embodiments, the wireless data communication can implement a predefined wireless communication protocol, such as the LoRaWAN protocol as shown in FIG. 2.

The edge gateway device 105 includes sensor data processing functionality 105B that is configured to collect, aggregate, and process the time-series data (i.e., the high-frequency location-specific raw methane concentration data and the high-frequency location-specific raw environmental data) communicated from the multiple networks of methane emission detectors 101, 103, for example, by filtering and/or averaging the data, to generate time-series operational data. The time-series operational data represents i) methane concentration at specific locations within the well site 100A as a function of time as derived from the location-specific raw methane concentration data from well site 100A, and ii) environmental conditions at specific location(s) within the well site 100B as a function of time as derived from the location-specific raw environmental data from well site 100A. The time-series operational data further represents i) methane concentration at specific locations within the well site 100B as a function of time as derived from the location-specific raw methane concentration data from well site 100B, and ii) environmental conditions at specific location(s) within the well site 100B as a function of time as derived from the location-specific raw environmental data from well site 100B The sensor data processing functionality 105B is further configured to cooperate with the Northbound Interface 105C to forward such time-series operational data to the remote cloud computing environment 109 via the data communication network 107. The cloud computing environment 109 receives the time-series operational data communicated from the edge gateway device 105 and processes the received time-series operational data for each well site to detect the presence of methane emission at the respective well sites 100A, 100B, the location of the methane emission at the respective well sites 100A, 100B (when present), and the associated rate of methane emission at the respective well sites 100A, 100B (when present). The cloud computing environment 109 can generate data related to the methane emission for the respective well sites 100A, 100B (such as the location of the methane emission for the respective well sites 100A, 100B, and the associated rate of methane emission for the respective well sites 100A, 100B) and process such data to automatically generate an alert characterizing methane emission for the respective well sites 100A, 100B. The alert can be communicated to a designated party or user to initiate or schedule mitigation of the methane emission at the respective well sites 100A, 100B.

In embodiments, the cloud computing environment 109 can employ suitable computational models, such as Gaussian plume dispersion models, to detect the presence of methane emission at the respective well sites 100A, 100B, the location of the methane emission at the respective well sites 100A, 100B (when present), and the associated rate of methane emission at the respective well sites 100A, 100B (when present). In this case, the computational models for the respective well sites 100A, 100B can be used as part of separate simulation and inversion operations that detect the presence of methane emission at the respective well sites 100A, 100B, the location of the methane emission at the respective well sites 100A, 100B (when present), and the associated rate of methane emission at the respective well sites 100A, 100B (when present).

In this manner, the edge gateway device 105 can be configured to collect and process time-series sensor data measured by methane emission detector networks at multiple industrial facilities (e.g., multiple well sites), and the cloud computing environment can be configured to process operational data derived from the time-series sensor data measured by the methane emission detector networks at the multiple industrial facilities to detect the presence of methane emission at the respective industrial facilities, the location of the methane emission at the respective industrial facilities (when present), and the associated rate of methane emission at the respective industrial facilities (when present).

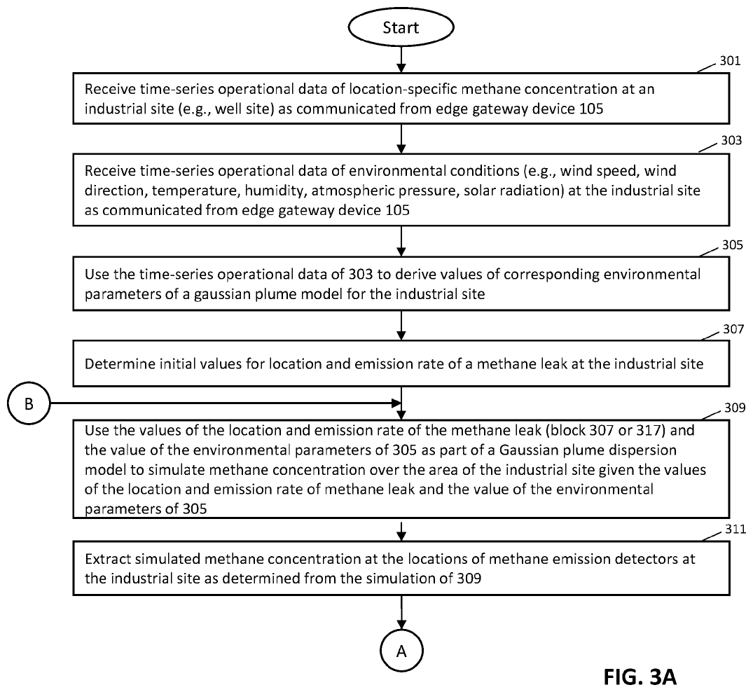

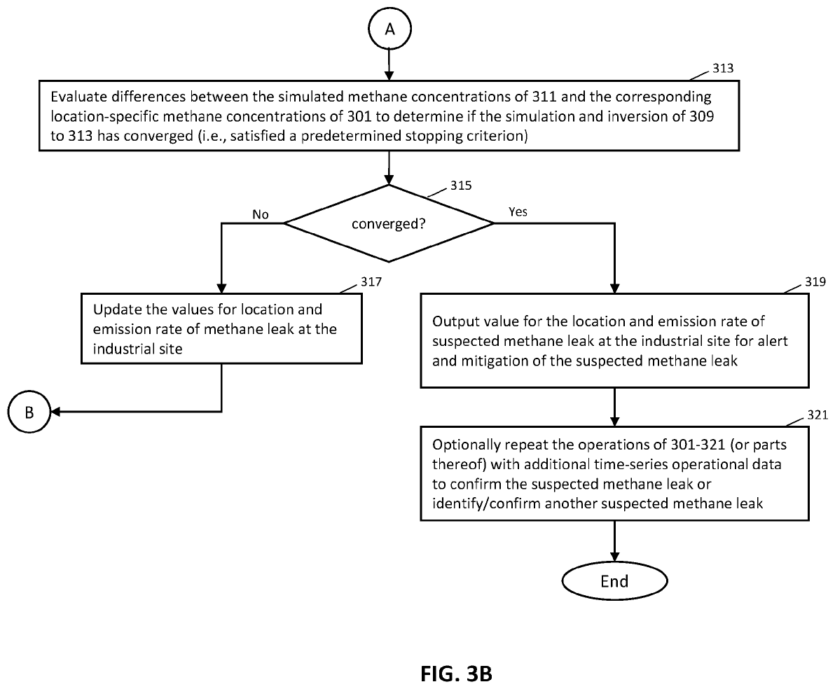

FIGS. 3A and 3B, collectively, is a flow chart of operations that can be carried out by the cloud computing environment 109 of FIGS. 1 and 2.

The operations begin in block 301 where the cloud computing environment 109 is configured to receive time-series operational data of location-specific methane concentration at an industrial site (e.g., a well site) as communicated from edge gateway device 105.

In block 303, the cloud computing environment 109 is configured to receive time-series operational data of location-specific environmental conditions (e.g., wind speed, wind direction, temperature, humidity, atmospheric pressure, solar radiation) at the industrial site as communicated from the edge gateway device 105.

In block 305, the cloud computing environment 109 is configured to use the time-series operational data of 303 to derive values of corresponding environmental parameters of a Gaussian plume dispersion model for the industrial site.

In block 307, the cloud computing environment 109 is configured to determine initial values for the location and emission rate of a methane leak at the industrial site.

In block 309, the cloud computing environment 109 is configured to use the values for the location and emission rate of the methane leak (block 307 or 317) and the value of the environmental parameters of 305 as part of a Gaussian plume dispersion model for the industrial site. The cloud computing environment 109 runs (or executes) the Gaussian plume dispersion model to simulate methane concentration over the area of the industrial site given the values for the location and emission rate of methane leak and the value of the environmental parameters of 305.

In block 311, the cloud computing environment 109 is configured to extract simulated methane concentration at the locations of methane emission detectors at the industrial site as determined from the simulation of 309.

In block 313, the cloud computing environment 109 is configured to evaluate differences between the simulated methane concentrations of 311 and the corresponding location-specific methane concentrations of 301 to determine if the simulation and inversion of 309 to 313 has converged (i.e., satisfied a predetermined stopping criterion).

In block 315, the cloud computing environment 109 determines if the simulation and inversion of 309 to 313 has converged. If not, the operations continue to block 317. If so, the operations continue to block 319 to 321.

In block 317, the cloud computing environment 109 is configured to update the values for the location and emission rate of methane leak at the industrial site, and the processing returns to block 309.

In block 319, the cloud computing environment 109 is configured to output values for the location and emission rate of suspected methane leak at the industrial site for alert and mitigation of the suspected methane leak. In embodiments, the last values for the location and emission rate of methane leak that resulted in convergence as determined in block 315 can be output as the values for the location and the emission rate of suspected methane leak at the industrial site.

In block 321, the cloud computing environment 109 can be configured to optionally repeat the operations of 301-321 (or parts thereof) with additional time-series operational data to confirm the suspected methane leak or identify/confirm another suspected methane leak.

In other embodiments, the methane emission detectors of the network(s) can be configured to process the real-time measurement data, for example, including filtering and/or averaging, and forward the resultant time-series data to the gateway device. The gateway device can collect, aggregate, and process such data for communication to the cloud computing environment.

In still other embodiments, the gateway device 105 can be configured to process time-series operational data derived from the measurements performed by the network(s) of methane emission detectors wirelessly coupled thereto using a suitable computational model, such as Gaussian plume dispersion model, to characterize methane emission at one or more industrial facilities. For example, the gateway device can process the time-series operational data using a suitable computational model (e.g., simulation and inversion using a Gaussian plume dispersion model) to detect the presence of methane emission at one or more industrial facilities, the location of the methane emission at one or more industrial facilities (when present), and the associated rate of methane emission at the one or more industrial facilities (when present). The gateway device can be further configured to generate data related to the methane emission (such as the location of the methane emission at the one or more industrial facilities, and the associated rate of methane emission at the one or more industrial facilities) and process such data to automatically generate alert(s) characterizing the methane emission at the one or more industrial facilities. The alert(s) can be communicated to designated party(ies) or user(s) to initiate or schedule mitigation of the methane emission at the one or more industrial facilities.

FIG. 4 depicts the automated system for methane emissions monitoring of FIG. 1 deployed at a well site (e.g., well pad).

FIG. 5 depicts the automated system for methane emissions monitoring of FIG. 1 deployed at another well site (e.g., well pad).

In embodiments, the methods and systems of the present disclosure as described herein may be adapted to utilize a wireless mesh sensor network. In general, wireless data communication between the methane emission detectors and the gateway that employs the LoRaWAN protocol will have approximately a maximum 10-mile reach in terms of distance between the methane emission detectors and the gateway. In areas with hills/trees, this maximum distance may be reduced. In embodiments, the methods and systems of the present disclosure may configure the methane emission detectors as a wireless mesh sensor network. In a wireless mesh sensor network, the methane emission detectors employ a wireless communication protocol to route the time-series operational data (e.g., time-series location-specific methane concentration data, time-series location-specific environmental condition data and detector-specific acceleration data derived from the measurements performed by methane emission detectors) to the gateway. Different from a star network where each methane emission detector communicates directly to the gateway device, the methane emission detectors of the wireless mesh sensor network pass the time-series operational data to each other, acting as a repeater for other detectors when necessary. One or more methane emitters of the wireless mesh sensor network can be configured for direct wireless communication to the gateway device. In embodiments, the methane emission detectors of the wireless mesh sensor network can be configured to send the time-series operational data to the nearest methane emission detector, which forwards such data onwards for communication to the gateway device. The idea is that the data hops to the nearest neighbor that it can communicate with but will ultimately reach the gateway device.

The wireless mesh sensor network is advantageous for applications where one or more methane emission detectors are located far away from the gateway device and cannot communicate directly with the gateway device but can communicate with a nearby detector. For example, in applications where methane emission detectors for multiple well pads are located at a distance (e.g., generally greater than 10 miles for LORAWAN communication) that makes direct wireless communication to the gateway device difficult or impossible, the wireless mesh sensor network enables efficient wireless communication from the methane emission detectors to the gateway device by hopping from one methane emission detector to the next, and the data transmitting via those detectors to the gateway device. In this configuration, the methane emission detectors create a mesh which implies that the data can hop from one methane emission detector to a nearby detector.

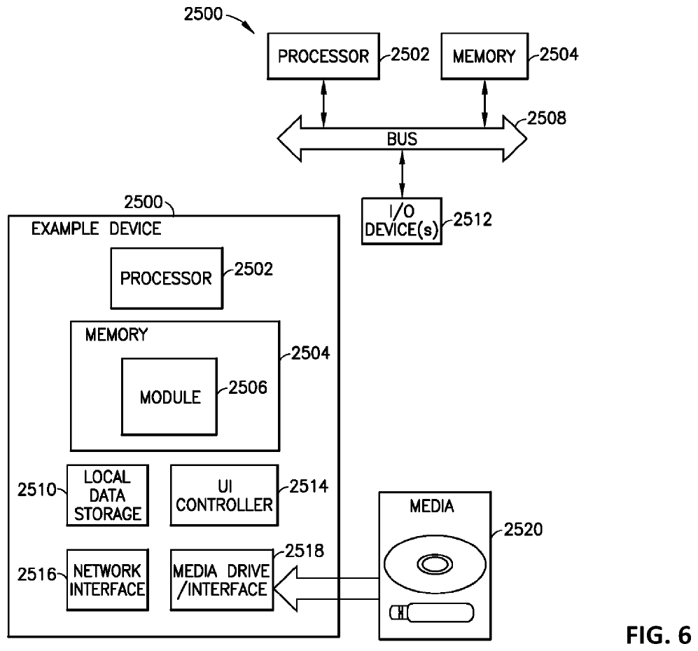

In some embodiments, the methods and system of the present disclosure may involve a computing system. FIG. 6 illustrates an example computing system 2500, with a processor 2502 and memory 2504 that can be configured to implement various embodiments of the automated systems and methods for methane emissions monitoring as discussed in the present application. Memory 2504 can also host one or more databases and can include one or more forms of volatile data storage media such as random-access memory (RAM), and/or one or more forms of nonvolatile storage media (such as read-only memory (ROM), flash memory, and so forth).

Device 2500 is one example of a computing device or programmable device and is not intended to suggest any limitation as to scope of use or functionality of device 2500 and/or its possible architectures. For example, device 2500 can comprise one or more computing devices, programmable logic controllers (PLCs), etc.

Further, device 2500 should not be interpreted as having any dependency relating to one or a combination of components illustrated in device 2500. For example, device 2500 may include one or more of computers, such as a laptop computer, a desktop computer, a mainframe computer, etc., or any combination or accumulation thereof.

Device 2500 can also include a bus 2508 configured to allow various components and devices, such as processors 2502, memory 2504, and local data storage 2510, among other components, to communicate with each other.

Bus 2508 can include one or more of any of several types of bus structures, including a memory bus or memory controller, a peripheral bus, an accelerated graphics port, and a processor or local bus using any of a variety of bus architectures. Bus 2508 can also include wired and/or wireless buses.

Local data storage 2510 can include fixed media (e.g., RAM, ROM, a fixed hard drive, etc.) as well as removable media (e.g., a flash memory drive, a removable hard drive, optical disks, magnetic disks, and so forth). One or more input/output (I/O) device(s) 2512 may also communicate via a user interface (UI) controller 2514, which may connect with I/O device(s) 2512 either directly or through bus 2508.

In one possible implementation, a network interface 2516 may communicate outside of device 2500 via a connected network. A media drive/interface 2518 can accept removable tangible media 2520, such as flash drives, optical disks, removable hard drives, software products, etc. In one possible implementation, logic, computing instructions, and/or software programs comprising elements of module 2506 may reside on removable media 2520 readable by media drive/interface 2518.

In one possible embodiment, input/output device(s) 2512 can allow a user (such as a human annotator) to enter commands and information to device 2500, and also allow information to be presented to the user and/or other components or devices. Examples of input device(s) 2512 include, for example, sensors, a keyboard, a cursor control device (e.g., a mouse), a microphone, a scanner, and any other input devices known in the art. Examples of output devices include a display device (e.g., a monitor or projector), speakers, a printer, a network card, and so on.

Various systems and processes of present disclosure may be described herein in the general context of software or program modules, or the techniques and modules may be implemented in pure computing hardware. Software generally includes routines, programs, objects, components, data structures, and so forth that perform particular tasks or implement particular abstract data types. An implementation of these modules and techniques may be stored on or transmitted across some form of tangible computer-readable media. Computer-readable media can be any available data storage medium or media that is tangible and can be accessed by a computing device. Computer readable media may thus comprise computer storage media. “Computer storage media” designates tangible media, and includes volatile and non-volatile, removable, and non-removable tangible media implemented for storage of information such as computer readable instructions, data structures, program modules, or other data. Computer storage media include, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other tangible medium which can be used to store the desired information, and which can be accessed by a computer.

Some of the methods and processes described above, can be performed by a processor. The term “processor” should not be construed to limit the embodiments disclosed herein to any particular device type or system. The processor may include a computer system. The computer system may also include a computer processor (e.g., a microprocessor, microcontroller, digital signal processor, general-purpose computer, special-purpose machine, virtual machine, software container, or appliance) for executing any of the methods and processes described above.

The computer system may further include a memory such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device.

Alternatively or additionally, the processor may include discrete electronic components coupled to a printed circuit board, integrated circuitry (e.g., Application Specific Integrated Circuits (ASIC)), and/or programmable logic devices (e.g., a Field Programmable Gate Arrays (FPGA)). Any of the methods and processes described above can be implemented using such logic devices.

Some of the methods and processes described above, can be implemented as computer program logic for use with the computer processor. The computer program logic may be embodied in various forms, including a source code form or a computer executable form. Source code may include a series of computer program instructions in a variety of programming languages (e.g., an object code, an assembly language, or a high-level language such as C, C++, or JAVA). Such computer instructions can be stored in a non-transitory computer readable medium (e.g., memory) and executed by the computer processor. The computer instructions may be distributed in any form as a removable storage medium with accompanying printed or electronic documentation (e.g., shrink wrapped software), preloaded with a computer system (e.g., on system ROM or fixed disk), or distributed from a server or electronic bulletin board over a communication system (e.g., the Internet or World Wide Web).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

What is claimed is:

1. A system for monitoring methane emissions at one or more industrial facilities, the system comprising:
a network of methane emission detectors spaced from one another at different locations within at least one industrial facility;
a gateway device operably coupled to the network of methane emission detectors; and
a cloud computing environment operably coupled to the gateway device;
wherein the network of methane emission detectors is configured to perform time-series measurements at different locations within the at least one industrial facility and wirelessly communicate time-series sensor data representing such measurements to the gateway device;
wherein the gateway device is configured to process the time-series sensor data to derive time-series operational data and communicate the time-series operational data to the cloud computing environment; and
wherein the cloud computing environment is configured to receive and process the time-series operational data to detect and characterize methane emission at the at least one industrial facility.

2. The system of claim 1, wherein:
the cloud computing environment is configured to process the time-series operational data in conjunction with a computational model to determine the location of methane emission at the at least one industrial facility and the associated rate of methane emission at the at least one industrial facility.

3. The system of claim 1, wherein:
the time-series operational data represents methane concentration at specific locations within the at least one industrial facility as a function of time and environmental conditions at one or more specific locations within the at least one industrial facility as a function of time; and
wherein the cloud computing environment is configured to process such time-series operational data in conjunction with a computation model that simulates methane emission at the at least one industrial facility based on environmental conditions within the at least one industrial facility.

4. The system of claim 2 or 3, wherein:
the processing of the time-series operational data involves inversion operations that evaluate differences between the time-series operational data and corresponding simulated methane concentration derived from the computation model.

5. The system of claim 2 or 3 or 4, wherein:
the computational model comprises a Gaussian plume dispersion model.

6. The system of claim 1, wherein:
each methane emission detector of the network wirelessly communicates time-series sensor data directly to the gateway device using a predefined wireless communication protocol such as the LORAWAN protocol; or
the network of methane emission detectors is configured as a wireless mesh sensor network wherein time-series sensor data is wirelessly communicated to a nearby methane emission detector for forwarding to the gateway device.

7. The system of claim 1, wherein:
the cloud computing environment is further configured to generate data related to the methane emission and process such data to automatically generate an alert characterizing the methane emission at the least one industrial facility.

8. The system of claim 1, wherein:
the at least one industrial facility comprises an oil and gas facility such as a well site, compressor stations, or processing facilities.

9. The system of claim 1, wherein:
the gateway device is configured to collect and process time-series sensor data measured by methane emission detectors at multiple industrial facilities; and
the cloud computing environment is configured to process operational data derived from the time-series sensor data measured by the methane emission detectors at the multiple industrial facilities to characterize methane emission at the respective industrial facilities.

10. The system according to claim 9, wherein:
the multiple industrial facilities comprises multiple oil and gas facilities such as one or more well sites, one or more compressor stations, or one or more processing facilities.

11. A system for monitoring methane emissions at one or more industrial facilities, the system comprising:
a network of methane emission detectors spaced from one another at different locations within at least one industrial facility; and
a gateway device operably coupled to the network of methane emission detectors;
wherein the network of methane emission detectors is configured to perform time-series measurements at different locations within the at least one industrial facility and wirelessly communicate time-series sensor data representing such measurements to the gateway device; and
wherein the gateway device is configured to process the time-series sensor data to derive time-series operational data and process the time-series operational data to detect and characterize methane emission at the at least one industrial facility.

12. The system of claim 11, wherein:
the gateway device is configured to process the time-series operational data in conjunction with a computational model to determine the location of methane emission at the at least one industrial facility and the associated rate of methane emission at the at least one industrial facility.

13. The system of claim 11, wherein:
the time-series operational data represents methane concentration at specific locations within the at least one industrial facility as a function of time and environmental conditions at one or more specific locations within the at least one industrial facility as a function of time; and
wherein the gateway device is configured to process such time-series operational data in conjunction with a computation model that simulates methane emission at the at least one industrial facility based on environmental conditions within the at least one industrial facility.

14. The system of claim 12 or 13, wherein:
the processing of the time-series operational data involves inversion operations that evaluate differences between the time-series operational data and corresponding simulated methane concentration derived from the computation model.

15. A system of claim 12 or 13 or 14, wherein:
the computational model comprises a Gaussian plume dispersion model.

16. The system of claim 11, wherein:
each methane emission detector of the network wirelessly communicates time-series sensor data directly to the gateway device using a predefined wireless communication protocol such as the LORAWAN protocol; or
the network of methane emission detectors is configured as a wireless mesh sensor network wherein time-series sensor data is wirelessly communicated to a nearby methane emission detector for forwarding to the gateway device.

17. The system of claim 11, wherein:
the gateway device is further configured to generate data related to the methane emission and process such data to automatically generate an alert characterizing the methane emission at the at least one industrial facility.

18. The system of claim 11, wherein:
the at least one industrial facility comprises an oil and gas facility such as a well site, compressor stations, or processing facilities.
